(19) United States
(12) Patent Application Publication
(10) Pub. No.: US 2005/0027333 A1
(43) Pub. Date: Feb. 3, 2005

(54) Methods and systems for monitoring range of motion for a patient's head and neck area

(76) Inventor: Arlene J. Lennox, Elburn, IL (US)

Correspondence Address: Luis M. Ortiz;Ortiz & Lopez, PLLC, P.O. Box 4484, Albuquerque, NM, 87196, US

(21) Appl. No.: 10/638,827

(22) Filed: Aug. 11, 2003

Related U.S. Application Data
(60) Provisional application No. 60/407,093, filed on Aug. 30, 2002.

(51) Int. Cl. A61N 1/00

(52) U.S. Cl. 607/89

(57) ABSTRACT

Methods and systems monitor a patient's mobility and range-of-motion (ROM) for a patient's head and neck area. Monitoring can be provided prior to, during and after associated therapy. At least one large protractor having an outer and an inner surface and including a circular scale including a vertex located along said at least one large protractor's inner surface can be selectively suspended above and in front of a patient measurement location. The at least one large protractor can be adapted for equidistantly positioning a patient's head away with respect to said circular scale of said at least one large protractor in a manner that the patient is generally centered and positioned comfortably in front or beneath the vertex of said at least one large protractor. A head mountable harness including securing means adapted for temporary attachment of a laser about said head mounted harness. At least one laser can be mounted about the head mountable harness and is capable of illuminating a laser beam onto said circular scale. The laser's beam is used to indicate the range of motion of the patient's head based on said laser beam's location indicated along said circular scale.

### PRIORITY TO PREVIOUSLY FILED APPLICATION

This application claims the benefit of priority to a provisional patent application, Ser. No. 60,407,093, filed on Aug. 30, 2002, and entitled “methods and systems for administering and monitoring results of microcurrent therapy administered to cancer patients for treatment and prevention of side effects associated with cancer treatment.”

### STATEMENT OF GOVERNMENT INTEREST

The United States government has certain rights to the present disclosure in accordance with contract DE-AC02-76CH03000 with the U.S. Department of Energy.

### FIELD OF THE INVENTION

The present invention generally relates to the monitoring of treatments addressing the side effects of cancer therapy. More particularly, the present invention is related to systems and methods for measuring a patient's mobility before, during and after the treatment and prevention of many medical conditions associated with cancer using radiation, chemotherapy and surgery, including: radiation-induced fibrosis; scar tissue; trismus; limited range of motion; loss of motor coordination; edema; and lymphedema. The present invention also related to the acquisition of patient mobility data before, during and after cancer therapy.

### BACKGROUND OF THE INVENTION

Over the years treatment schemes for head and neck malignancies have become progressively aggressive, combining cytotoxic chemotherapy agents with escalating doses of radiation, and sometimes surgery. Despite increasing tumor control, treatment-induced quality-of-life sequelae remain problematic. As aggressive therapy with combination surgery, chemotherapy and radiation increases tumor control in head-and-neck neoplasms, post-treatment quality of life issues remain problematic. One area of concern is over progressive fibrosis of soft tissue in the head, neck and supraclavicular area that can result from such therapy. For many patients, palpation of the treated areas reveals hard, unyielding tissue that limits range of motion and/or leads to pain associated with movement. Patients often experience limited mobility and range of motion in areas affected by disease, or as a result of treatment. Limited mobility and range of motion should be monitored before during and after treatment in order to assess it effectiveness. The following treatments exemplify treatments that have shown an effect on patient mobility.

Radiation therapy uses penetrating beams of radiation to treat or remediate disease. Various forms of radiation therapy, including photon, electron, and neutron radiation, are used on a daily basis in the United States and throughout the world. One major use of radiation therapy is in the treatment of cancerous tumors. The basic effect of radiation therapy is to destroy the ability of cells to divide and grow by damaging their DNA strands. This effect is useful in killing cancerous cells, but also has the major disadvantage of damaging healthy tissue. As a result, a patient may be required to live with debilitating side effects of cancer treatment including limb or organ swelling, thickening and hardening of otherwise normal tissue, and chronic or constant pain.

The deleterious side effects produced in the patient as a result of radiation therapy are known as radiation toxicities. Radiation toxicities are associated with any ionizing radiation treatment and include fibrotic tissue (scar tissue), xerostomia (loss of salivary function), trismus (closure of the jaw), radiation proctitis (inflammation of the rectum), limited range of motion, loss of motor coordination, edema (swelling), and lymphedema (swelling resulting from obstruction of the lymphatic vessels or lymph nodes). Unfortunately, side effects associated with radiation therapy are progressive and in most cases will tend to worsen over time. Current practices for treating late side effects of cancer treatment include physical therapy, massage, exercise, and drugs such as diuretics, painkillers, steroids, and saliva inducers. In most cases, however, such treatments can only provide patients with minimal relief, yet patients will most likely be required to live with the debilitating side effects of radiation therapy or, unfortunately, chemical and/or biological side affects from medicinal therapies used to treat radiation side effects.

Use of electrical stimulation for simple pain relief, not associated with cancer or radiation therapy, has been well established by physical therapy centers. Physical therapists use microcurrent therapy in a variety of ways for the treatment of pain not associated with cancer or radiation treatment, often in combination with massage, heat and physical manipulation. There are many commercially electrical stimulation devices marketed for the treatment of pain relief, most of which are commonly referred to as TENS (transcutaneous electrical nerve stimulation) units. Typical TENS units emit electrical pulses with alternating positive and negative polarities in the 10 to 500 kilohertz (KHz) range and currents in the milliampere (mA) range. Microcurrent (μA) units are often incorrectly referred to as TENS units, but microcurrent units deliver lower currents (microampere range) and lower frequencies (0.5 to several hundred Hertz). In general, units using higher current and frequencies are more effective at blocking acute pain, but the pain relief is not lasting. By contrast, microcurrent therapy using lower frequencies requires longer treatment times to achieve pain relief, but the relief can endure for many hours after the treatment has terminated.

The present inventor has previously recognized that a need existed in the medical profession for a more effective means of alleviating radiation toxicities typically associated with radiation therapy. In U.S. Pat. No. 6,115,637, entitled “microcurrent therapeutic technique for treatment of radiation toxicity,” issued Sep. 5, 2000, and herein incorporated by reference, the present inventor previously disclosed methods for alleviating radiation toxicities associated with radiation therapy. As described in the patent, a sinusoidally pulsed biphasic DC signal can be applied to an affected bodily area using at least one electrode. The electrode can be manipulated using active tactile manipulation for a predetermined time and the frequency of the sinusoidally pulsed biphasic DC signal can be adjusted during the course of the treatment. The patent also describes a method of applying a spiked pulsed biphasic DC signal to an affected bodily area using at least one electrode. The electrode can also be manipulated using active tactile manipulation for a predetermined time and the frequency of the spiked pulsed biphasic DC signal can also be adjusted during the course of the treatment.

The microcurrent therapeutic techniques described in the '637 patent are used to alleviate debilitating radiation toxicities associated with radiation therapy, including fibrotic tissue, xerostomia, trismus, radiation proctitis, limited range of motion, loss of motor coordination, edema, and lymphedema. An objective set forth in the '637 patent is to provide greater pain relief than prior known methods for treating the late side effects of cancer treatment, thus allowing patients to have higher qualities of life. The patent also describes use of methods for pre-treating a patient in an effort to avoid the radiation toxicities associated with radiation therapy.

Since disclosing the patented use of microcurrent therapy treatment of toxicities associated with radiation cancer treatment, the present inventor has determined that a need exists for treatment regimes that include monitoring. Treatment monitoring can be used to accurately target and treat, pre-treat, and concurrently treat areas on the human body that fall ill as a result of cancer therapies or show the need for additional treatment. The present inventor therefore now discloses new methods and systems for monitoring mobility in patients. Monitoring can be conducted at and during initial diagnosis, pretreatment, concurrent treatment and ongoing treatment. Side effects of cancer-specific treatment that would benefit from monitoring include: radiation-induced fibrosis; trismus; limited range of motion; loss of motor coordination; edema, and lymphedema.

### SUMMARY OF THE INVENTION

The following summary of the invention is provided to facilitate an understanding of some of the innovative features unique to the present invention and is not intended to be a full description. A full appreciation of the various aspects of the invention can only be gained after considering the entire specification, claims, drawings and abstract as a whole.

Microcurrent therapy has shown promise in improving range-of-motion in patients experiencing discomfort and mobility issues, and also for alleviating symptoms associated with certain medical therapies, such as radiation therapy-induced fibrosis. Protocols combining microcurrent therapy with physical therapy and/or promising medications could prove to be very beneficial in improving a patient's quality of life. Patient improvement must be monitored in order to measure the effectiveness of treatments and therapies.

The present inventor has developed a range-of-motion (ROM) measuring system and methods for using the system to enabling the assessment of a patient's mobility condition and provide improvement monitoring.

Accordingly, it is an aspect of the present invention to provide a system and methods for measuring the range-of-motion for a patient's head and neck area.

It is another aspect of the present invention to provide a system and methods for obtaining measurements of at least one of cervical rotation, extension/flexion, and lateral flexion.

It is yet another aspect of the present invention to provide a system and methods that enables periodic (e.g., daily, weekly, monthly) patient positioning accuracy of, for example, about ±0.25 cm based on a scale that is located about 112 cm away from the patient.

It is yet another aspect of the present invention to allow for a choice of scale size that can help minimize the effect of errors that can occur from re-positioning a patient's center of rotation at the vertex of the system's measurement scale.

It is yet another aspect of the present invention to provide a methods for diagnosing patient recovery or condition using the present invention.

In accordance with features of the present invention, cervical rotation, extension/flexion and lateral flexion can be measured using a system that can include two large protractors mounted in perpendicular planes above a patient location. A head mounted harness, which can include an elastic band with Velcro attachments, can be secured to a patient's head and can enable at least one small laser to be mounted to the harness. The laser is mounted on the harness in such a manner that is can be used to point to unit markings (e.g., units of degrees) located on circular scales associated with the protractors. Movement of a patient's head causes movement of the laser's beam along the protractors, thereby allowing for the measurement of a patient's ROM in units, which can preferably be provided in units of degrees.

During use of the measurement system, a laser should be positioned relative to the point(s) about which the patient's head pivots during rotation, extension/flexion and lateral flexion. Additional stationary lasers can be used to position the patient in such a position under the protractors that the head-mounted movable laser(s) is/are on a line that intersects the vertex of the large protractors.

Periodic, e.g., daily, patient positioning accuracy of about ±0.25 cm can be achieved using the present measuring system, which is a small error span compared to the protractors' radius from the patient's head, which can be about 112 cm. This choice of scale size can minimize the effect of errors that can occur from re-positioning a patient's center of rotation at the vertex of the system's measurement scale.

Diagnosis of patient recovery or condition can be determined using the present invention. For example, pretreatment data used to classify each range of motion can be classified as asymptomatic, mildly limiting, moderately limiting or severely limiting. If a patient's range falls within 90% of the optimal range for a healthy young person he or she can be classified as asymptomatic for that measurement. Ranges between 70 and 90% of optimum can be designated as mildly limiting, while 50-70% can be designated as moderately limiting. Ranges less than 50% of optimum can be considered severely limiting. By assigning a value of 0 to asymptomatic, 1 to mild, 2 to moderate, and 3 to severe, for each of three range-of-motion measurements it is possible for therapists to assign a number between 0 and 9 to each patient, with 0 corresponding to no practical limitations and 9 corresponding to significant limitations in all three measurements that are enabled with the present invention.

### BRIEF DESCRIPTION OF THE DRAWINGS

The accompanying figures, in which like reference numerals refer to identical or functionally-similar elements throughout separate views and which are incorporated in and form part of the specification, further illustrate the present invention and together with the detailed description of the invention serve to explain the principles of the present invention.

FIG. 1(6) is an illustration of a measurement system wherein a patient is positioned at the vertex of two mutually perpendicular protractors used to measure cervical range of motion;

FIG. 2(7) is an illustration of a measurement system wherein a laser is affixed near a patient's head to measure left-right cervical rotation;

FIG. 3(8) is an illustration of a measurement system wherein cervical extension/flexion is being measured using a laser affixed near the side of a patient's head;

FIG. 4(9) is an illustration of a measurement system wherein cervical lateral flexion is being measured using a laser affixed near a patient's forehead;

FIG. 5(13) is a graphical illustration of the range of cervical rotation over time for three microcurrent therapy patients initially experiencing severe range-of-motion limitations;

FIG. 6(14) is a graphical illustration of the range of cervical extension/flexion over time for three microcurrent therapy patients initially experiencing severe range-of-motion limitations; and

FIG. 7(15) is a graphical illustration of range of cervical lateral flexion over time for four microcurrent therapy patients initially experiencing severe range-of-motion limitations.

### DETAILED DESCRIPTION

The particular values and configurations discussed in these nonlimiting examples can be carried and are cited merely to illustrate at least one embodiment of the present invention and are not intended to limit the scope of the invention.

Microcurrent therapy has shown promise in improving range-of-motion in patients experiencing discomfort and mobility issues, and also for alleviating symptoms associated with certain medical therapies, such as radiation therapy-induced fibrosis. Protocols combining microcurrent therapy with physical therapy and/or promising medications could prove to be very beneficial in improving a patient's quality of life. Patient improvement must be monitored in order to measure the effectiveness of treatments and therapies.

The present inventor has developed a range-of-motion (ROM) measuring system and methods for using the system to enabling patient mobility condition assessment and improvement monitoring. Measurement and monitoring can begin at patient intake and continue through the acquisition of periodic measurements during therapy, such as at the end of each microcurrent treatment. Objective ROM measurements can be carried out for cervical rotation, extension/flexion, and lateral flexion before therapy, during and after therapy using the monitoring system as will now be further described.

As aggressive therapy with combination surgery, chemotherapy and radiation therapy increases tumor control in head-and-neck neoplasms, post-treatment quality of life issues remain problematic. One area of concern is progressive fibrosis of soft tissue in the head, neck and supraclavicular area. For many patients, palpation of the treated areas reveals hard, unyielding tissue that limits range of motion and/or leads to pain associated with movement.

Microcurrent therapy shows promise in improving range-of-motion and alleviating other symptoms associated with radiation-induced fibrosis. Protocols combining microcurrent therapy with physical therapy and/or promising medications could prove to be very beneficial in improving quality-of-life for radiation therapy patients.

Patients who experience late effects of radiation therapy can be treated with microcurrent therapy systems and methods. Objective range-of-motion (ROM) measurements can be carried out for cervical rotation, extension/flexion, and lateral flexion before therapy, during and after therapy using the monitoring system described herein. The present inventor has developed methods of treatment using microcurrent systems and has also developed and used a measuring system to monitor patient improvement at the end of each microcurrent treatment. Treatment can preferably be provided using impedance-controlled systems.

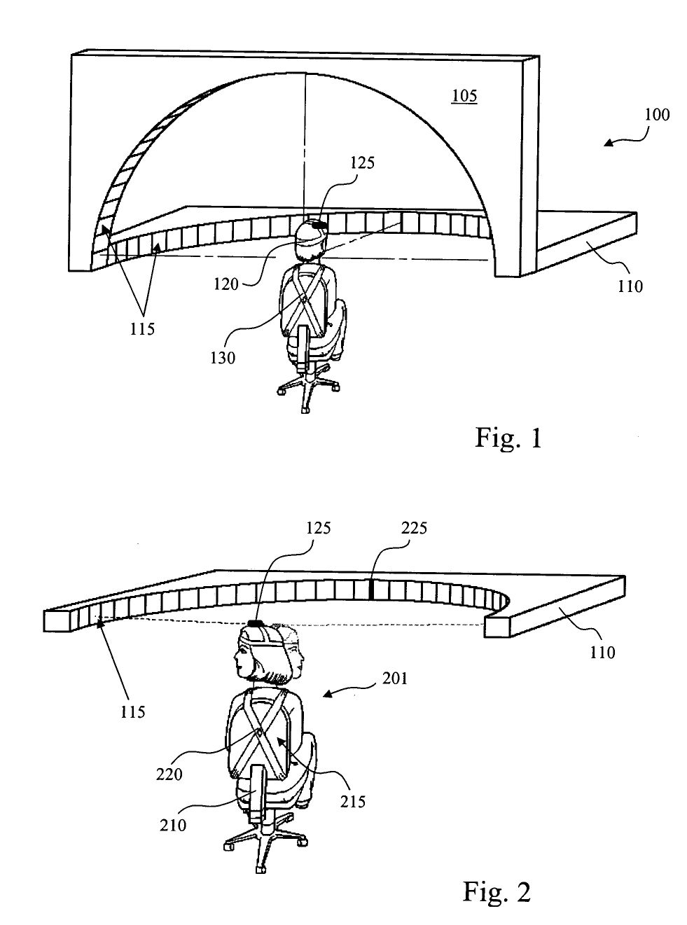

Referring to FIG. 1, cervical rotation, extension/flexion and lateral flexion can be measured using a system 100 that can include at least two large protractors, indicated by numerals 105 and 110, and shown mounted in perpendicular planes. A head mounted harness 120 can include an elastic band with Velcro attachments. The harness can be secured to the patient's head and to enable at least one small laser 125 mounted to the harness 120 to be pointed to markings 115 located on the protractors 105 and 110 as circular scales and used to measure range of motion for a patient's head based on the position of the laser beam (not shown) emanating from the laser 125 onto the markings 115.

During use of the measurement system 100, the laser 125 should be positioned relative to the points along the circular scales 115 about which the patient's head will pivot during its rotation, extension-flexion and lateral-flexion. It should be appreciate that more than one laser, similar to laser 125, can be position about the harness to illuminate more than one point along the markings 115 used to measure mobility of a patient's head relative to the patient's stationary torso. Furthermore, it should be appreciated that additional lasers, such as stationary lasers, can be used to position the patient so that the head-mounted movable laser(s) is/are on a line that intersects the vertex of the measurement scale, such as the two large protractors 105 and 110.

Referring to FIG. 2, a patient 201 is shown sitting on a chair 210 in such a manner that the patient's torso is held firmly in a stationary position against the chair's back 215 by straps 220. It should be appreciated that any suitable base that may be deployed by those skilled in the art can be used to support a patient. The chair 210 is shown for illustrative purposes without the intent of limitation. It should be appreciated by those skilled in the art that a patient may not be able to sit and will be supported, for example, in a standing or laying position, or standing without being supported by a base.

Day to day patient positioning accuracy of, for example, ±0.25 cm can be achieved using a measuring system in accordance with the present invention, which is a small error span compared to the protractors' radius, which can be set at a distance of about 112 cm from the patient's head. This choice of scale size can minimize the effect of day-to-day errors in positioning a patient's center of rotation at the vertex, or null, 225 of the system's scale. It is of primary importance, however, that the patient's torso remain still and oriented at null, or zero degrees as represented by the protractors in FIGS. 1-4, with respect to the measurement scale in use.

As part of the setup for obtaining measurement of a patient's ability to rotate her head left and right is illustrated in FIG. 2, the laser 125 is mounted on top of the harness 120 in such a manner that the laser beam illuminate horizontally to its target 225 on the scale 110, while the patient is comfortably positioned at null. The front of the sitting patient's torso is shown facing towards the center, vertex, or null point, 225 of the marked scale represented by a darkened marking. The patient's ability to rotate her head can then be measured to the right and left along the scale by obtaining laser-illuminated measurements points on the scale at the farthest point of rotation comfortably achievable by the patient.

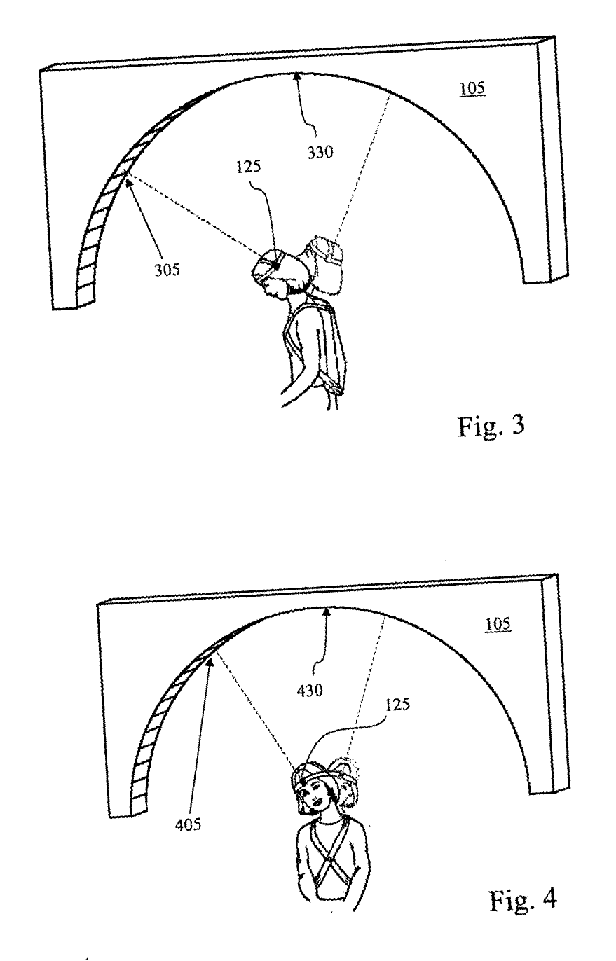

Referring to FIG. 3, a setup for obtaining measurement of a patient's ability to move her head forward and backward, or extension-flexion, is illustrated. The front of the sitting patient's torso is shown facing towards either end of the scale located above her, but generally located under the center of the marked scale. The laser 125 is positioned on the harness 120 in such a manner that the laser beam is illuminating vertically onto the vertex, or null point, 330 of the marked scale 305 used for this type of measurement. The patient's maximum ability to move her head forward and backward can then be measured along the scale 305 by obtaining laser-illuminated measurements points on the scale at the farthest point of movement the patient can comfortably achieve.

Referring to FIG. 4, a setup for obtaining measurement of a patient's ability to move her head side-to-side, or lateral-flexion, is illustrated. Compared to the set-up of FIG. 3, the patient is now situated under scale 405 such that the front of her torso is shown facing parallel along the ends of the scale 405 located above her. Again, the patient is generally located under the center of the marked scale, but this time the laser 125 can be positioned near the front of the harness 120 in such a manner that the laser beam will illuminate vertically onto the vertex, or null point, 430 of the marked scale 405 used for lateral-flexion measurement. The patient's maximum ability to move her head laterally by tilting it to hear left and right shoulders, can then be measured along the scale 405 by obtaining laser-illuminated measurements points at the farthest point of movement the patient can comfortably achieve laterally.

For patients, pretreatment data can be used to classify each range of motion as asymptomatic, mildly, moderately or severely limiting. If a patient's range was within 90% of the optimal range for a healthy young person he or she was classified as asymptomatic for that measurement. Ranges between 70 and 90% of optimum were designated mildly limiting, while 50-70% were moderately limiting. Ranges less than 50% of optimum were considered severely limiting. By assigning a value of 0 to asymptomatic, 1 to mild, 2 to moderate, and 3 to severe, for each of three range-of-motion measurements it is possible to assign a number between 0 and 9 to each patient, with 0 corresponding to no practical limitations and 9 corresponding to significant limitations in all three measurements. Using these designations, the average pretreatment severity for the 13 patients treated with photons only was 5.6±2.4.

In a clinical experiment conducted by the present inventor, treatment severity for 8 patients receiving only fast neutrons it was recorded at 4.0±2.7, and for 5 patients who were treated with neutrons following photon therapy a recording of 2.4±1.5 was obtained. Three patients having a severity of 9 had received electrons in addition to photons. Table 1 lists 26 cases studied by the present inventor in order of severity along with information about treatment site, tumor pathology, stage, type of radiation, and dose.

TABLE 1
Patient characteristics listed in order of greatest to least severe radiation-induced
range-of-motion limitations before impedance-controlled microcurrent therapy.

| | Radiation | Dose | | | | Other |
|---|---|---|---|---|---|---|
| Severity | Treatment Site | (Gy) | Radiation | Pathology | Stage | Therapy |
| 9 | Left thyroid | 66 | γ + e | Medullary | T4N1bM0/Stage 3 | Surgery |
| | Bilateral neck | 66 | γ + e | Carcinoma | | |
| | Supraclav nodes | | | | | |
| 9 | Oropharynx | 63 | γ + e | Squamous Cell | T1N2bM0 | Surgery |
| | Bilateral neck | | γ + e | | | |
| | Supraclav nodes | 50.4 | | | | |
| 9 | Left tonsil | 74.4* | γ + e | Squamous Cell | T3N2bM0 | Surgery |
| | Bilateral neck | | | | | Chemo |
| | Supraclav nodes | 50.4 | | | | |
| 8 | Nasopharynx | 22 | n | Squamous Cell | T2N2aM0/Stage 4 | |
| | Supraclav nodes | 14 | | | | |
| 7 | Maxillary sinus | 20.4 | n | Adenoid Cystic | T4NxM0 | Surgery |
| 6 | Supraglottic larynx | 75* | γ + e | Squamous Cell | T2N2bM0/Stage 4 | Chemo |
| | Supraclav nodes | 51 | | | | |
| 6 | Nasopharynx | 70 | | Squamous Cell | T2N2bM0/Stage 4 | Chemo |
| | Bilateral Neck | | γ | | | Surgery |
| | Supraclav nodes | 50 | | | | |
| 6 | Right Neck | 58.7 | | Colloidal | Metastatic from | Chemo |
| | Right supraclav | 45 | γ | Carcinoma | breast | |
| | nodes | | | | | |
| 6 | Nasopharynx & | 45 | γ | Malignant | Recurrent/Stage 4 | Chemo |
| | neck | | | lymphoma | | Surgery |
| | Periaortic nodes | | | | | |
| 6 | Larynx | 60.4 | γ + e | Squamous Cell | T4N0M0 | Surgery |
| | Bilateral neck | 50.4 | | | | |
| 5 | Right submaxillary | 20.4 | n | Adenoid cystic | Stage 1 | Surgery |
| 5 | Left Parotid | 22 | n | Adenoid cystic | T2N0M0/Stage 1 | Surgery |
| 4 | Left Parotid | 59.2 | γ | Melanoma | Metastatic from | Surgery |
| | | | | | cheek | |
| 4 | Left Parotid | 30 | γ | Benign mixed | Recurrent | Surgery |
| | | 20.4 | n | | | |
| 3 | Right nasal ala | 59.5 | γ + e | Squamous Cell | Recurrent | Surgery |
| | Bilateral neck | | | | | |
| | Supraclav nodes | 50.4 | γ | | | |
| 3 | Tongue | 60 | γ | Keratinizing | T2N1Mx | Surgery |
| | Left neck | 62.8 | γ + e | Squamous Cell | | |
| 3 | Base of tongue | 20 | n | Adenoid cystic | T1N0M0 | Surgery |
| 3 | Right | | | Adenoid cystic | T1N0Mx/Stage 1 | Surgery |
| | Submandibular | 7.2 | γ | | | |
| | | 20.4 | n | | | |
| | Right supraclav | 14.0 | n | | | |
| | nodes | | | | | |
| 3 | Left parotid | 19 | γ | Mucoepidermoid | T1N2bM0 | Surgery |
| | | 20.1 | n | | | |
| | Supraclav nodes | 14 | n | | | |
| 3 | Right tonsil | 74.4* | γ + e | Squamous Cell | T3N1M0 | Surgery |
| 2 | Left parotid | 20.8 | n | Acinic Cell | Recurrent | Surgery |
| | Left Supraclav | 14.3 | n | | | |
| | nodes | | | | | |
| 2 | Right tonsil | 61 | γ + e | Squamous Cell | T1N2bM0/Stage 4 | Surgery |
| | Bilateral neck | 64 | γ + e | | | |
| | Suprclav nodes | 46 | γ | | | |
| 2 | Left parotid | 60 | γ | Adenoid Cystic | Recurrent | Surgery |
| | | 20.4 | n | | | |
| 1 | Base of tongue | 20.4 | n | Mucoepidermoid | T3NxM0 | |
| 1 | Base of tongue | 20.4 | n | Adenoid Cystic | T4N1M0 | |
| 0 | Left parotid | 65 | γ | Adenoid Cystic | Recurrent | Surgery |
| | | 20.4 | n | | | |

*indicates bid treatment.

A microcurrent therapy system was used for delivering microcurrent up to about 600 microamps, at various frequencies (e.g., spiked/sinusoidal pulsed biphasic) in the range of 0.5 Hz to 500 Hz, and at various durations (e.g., about 20 minutes). A conductive gel was applied to the patient's neck and or head area in order to enhance conductivity. A sinusoidally pulsed biphasic DC signal was applied to the neck/had areas using a roller-type electrode, although it should be appreciated that other conductive electrodes can be used. The roller electrode should preferably be smooth enough, or rotate freely enough, to be maneuvered comfortably over the patient. The area targeted for treatment was the patient's neck area. The electrode (probe) can be manipulated over the patient's neck for a predetermined time. Generally a 10-20 minute treatment is within a comfortable range for most patients. Finally, the frequency of the sinusoidal signal can be adjusted during the course of treatment. Frequency can be varied within a 0.5-500 HZ range, and applied current during treatment can be up to about 600 microamps.

During the experiment, patients were treated with microcurrent therapy twice per day, with a four to five hour interval between treatment sessions. A total of ten treatments were given over a period of five days. Subjective symptoms were recorded and range-of-motion measurements were made before the first treatment and at the end of each treatment day. Follow-up measurements and subjective assessments were made at one-month intervals for a total of three months. No additional microcurrent or physical therapy was permitted until the end of the three-month follow-up period.

Tables 2-4 show the average pretreatment, post-treatment and 3-month follow-up ranges for cervical rotation, extension/flexion, and lateral flexion measurements stratified by pretreatment severity and type of radiation given. For each type of motion the degree of improvement is directly proportional to the pretreatment severity. Despite our expectations that any improvement observed at the end of the treatment week would be lost at the three-month follow-up visit, most patients had better measurements at three months than before treatment. At the three-month follow-up the average severity score for the photon-only patients was 3.9±2.3; for the neutron-only patients it was 1.2±1.2; and for the neutron-following-photon patients it was 2.0±1.0. No adverse side effects were observed. All of the patients completed the treatments.

The first three columns in Table 2 show the type of radiation received by the 26 patients who started the study, followed by the 22 patients who returned for the 3-month follow-up. Nn indicates number of patients who had only neutrons, Np is the number who received only photons. Np+n is the number of patients who were treated with neutrons after photon therapy. The total number in each category is given by N.

TABLE 2
Average cervical rotation ± standard deviation, stratified by severity of
limitation, before microcurrent treatment, at the end of treatment, and three months
later with no additional treatment. As shown in Optimal range-of-motion for a healthy
young person is 170 degrees.

| | | | | | | % Change | | % Change |
|---|---|---|---|---|---|---|---|---|
| | | | | Pretreatment | Posttreatment | from | 3-month | from |
| | | | Pretreatment | Range | Range | pretreatment | Follow-up | pretreatment |
| Nn | Np | Np + n | Rating | (degrees) | (degrees) | range | Range(degrees) | range |
| 1.0 | 3.3 | — | Severe | 59 ± 19 | 97 ± 30 | 64% | 83 ± 14 | 41% |
| | | | | N = 4 | N = 4 | | N = 3 | |
| 2.2 | 6.5 | 2.1 | Moderate | 101 ± 10 | 131 ± 15 | 30% | 119 ± 9 | 18% |
| | | | | N = 10 | N = 10 | | N = 8 | |
| 4.4 | 4.4 | 2.1 | Mild | 131 ± 8 | 153 ± 16 | 17% | 140 ± 13 | 7% |
| | | | | N = 10 | N = 10 | | N = 9 | |
| 1.1 | — | 1.1 | Asymptomatic | 164 ± 1 | 165 ± 9 | 1% | 154 ± 22 | −6% |
| | | | | N = 2 | N = 2 | | N = 2 | |

The first three columns in Table 3 show the type of radiation received by the 26 patients who started the study, followed by the 22 patients who returned for the 3-month follow-up. Nn indicates number of patients who had only neutrons, Np is the number who received only photons. Np+n is the number of patients who were treated with neutrons for a recurrence after photon therapy. The total number in each category is given by N.

TABLE 3
Average cervical extension/flexion ± standard deviation, stratified by
severity of limitation, before microcurrent treatment, at the end of treatment, and
three months later with no additional treatment. Optimal range-of-motion for a
healthy young person is 120 degrees.

| | | | | | | % Change | | % Change |
|---|---|---|---|---|---|---|---|---|
| | | | | Pretreatment | Posttreatment | from | 3-month | from |
| | | | Pretreatment | Range | Range | pretreatment | Follow-up | pretreatment |
| Nn | Np | Np + n | Rating | (degrees) | (degrees) | range | Range(degrees) | range |
| — | 3.3 | — | Severe | 47 ± 10 | 70 ± 12 | 49% | 73 ± 13 | 55% |
| | | | | N = 3 | N = 3 | | N = 3 | |
| 2.1 | 3.3 | — | Moderate | 73 ± 9 | 106 ± 9 | 45% | 107 ± 20 | 47% |
| | | | | N = 5 | N = 5 | | N = 4 | |
| 4.4 | 5.4 | 2.1 | Mild | 96 ± 7 | 114 ± 15 | 19% | 110 ± 9 | 15% |
| | | | | N = 11 | N = 11 | | N = 9 | |
| 2.2 | 2.2 | 3.2 | Asymptomatic | 117 ± 6 | 126 ± 15 | 8% | 117 ± 14 | 0% |
| | | | | N = 7 | N = 7 | | N = 6 | |

The first three columns of Table 4 show the type of radiation received by the 26 patients who started the study, followed by the 22 patients who returned for the 3-month follow-up. Nn indicates number of patients who had only neutrons, Np is the number who received only photons. Np+n is the number of patients who were treated with neutrons after photon therapy. The total number of patients in each category is given by N.

TABLE 4
Average cervical lateral flexion ± standard deviation, stratified by severity
of limitation, before microcurrent treatment, at the end of treatment, and three
months later with no additional treatment. Optimal range-of-motion for a healthy
young person is 90 degrees.

| | | | | | | % Change | | % Change |
|---|---|---|---|---|---|---|---|---|
| | | | | Pretreatment | Posttreatment | from | 3-month | from |
| | | | Pretreatment | Range | Range | pretreatment | Follow-up | pretreatment |
| Nn | Np | Np + n | Rating | (degrees) | (degrees) | range | Range(degrees) | range |
| 1.0 | 5.4 | — | Severe | 31 ± 7 | 51 ± 20 | 65% | 48 ± 9 | 55% |
| | | | | N = 6 | N = 6 | | N = 4 | |
| 2.2 | 4.4 | 1.1 | Moderate | 53 ± 5 | 76 ± 10 | 43% | 79 ± 16 | 49% |
| | | | | N = 7 | N = 7 | | N = 7 | |
| 3.3 | 4.4 | 1.1 | Mild | 69 ± 5 | 82 ± 17 | 19% | 75 ± 12 | 9% |
| | | | | N = 8 | N = 8 | | N = 8 | |
| 2.2 | — | 3.1 | Asymptomatic | 92 ± 22 | 102 ± 25 | 11% | 103 ± 30 | 12% |
| | | | | N = 5 | N = 5 | | N = 3 | |

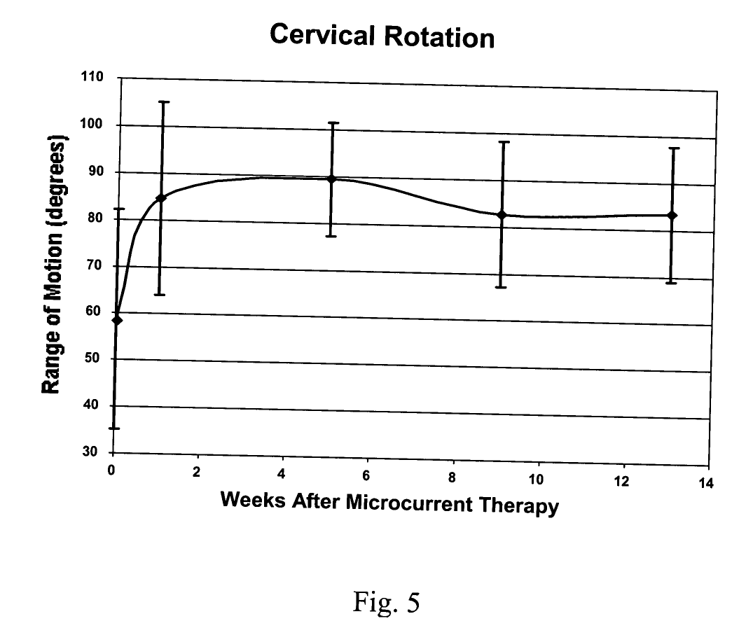

Referring to FIG. 5, a graph illustrates measured improvements for the three patients who started with severe limitations and completed all three follow-up visits on schedule. The range of right/left cervical rotation was compared to the nominal value of 170 degrees, which is considered normal for a healthy young individual. Ninety-two percent (24/26) of the patients exhibited improved cervical rotation at the end of microcurrent therapy. Of the twenty-two who returned for the three-month follow-up visit, three experienced continued improvement, while seventeen lost some of their range-of-motion, though their average mobility was somewhat better than it had been before microcurrent therapy. One patient in the mildly limited category experienced no improvement and one asymptomatic patient had measurements in the mildly limited category at.

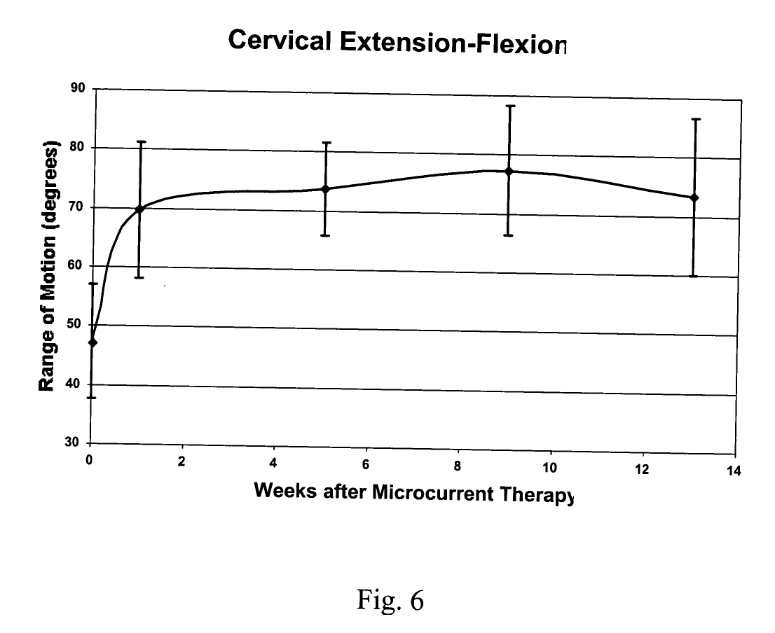

Referring to FIG. 6, a graph illustrates improvements for the three patients initially classified as most severely limited in extension/flexion. Range of cervical extension/flexion was compared to the nominal value of 120 degrees, which is considered normal for a healthy young individual. Eighty-five percent (22/26) of the patients exhibited improved extension/flexion at the end of microcurrent therapy. Of the twenty-two who returned for the three-month follow-up visit, eight maintained or improved their end-of-treatment status. Ten of the twenty-two patients lost some range of motion but their mobility was still better than it had been before microcurrent therapy. The four patients who experienced no long-term improvement were already functioning within 80-90% of normal range.

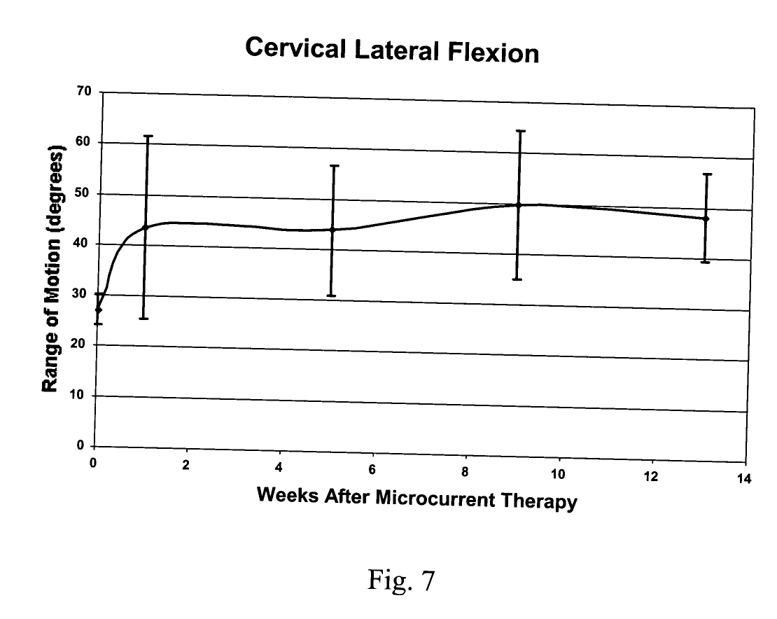

Referring to FIG. 7, a graph illustrates the improvements for the four patients who started with severe limitations and completed all three follow-up visits on schedule. Range of cervical right/left lateral flexion was compared to the nominal value of 90 degrees, which is considered normal for a healthy young individual. Eighty-one percent (21/26) of the patients exhibited improved range of lateral flexion at the end of microcurrent therapy. Of the twenty-two patients who returned for the three-month follow-up visit eight had continued to improve their range of motion without any additional therapy. Nine patients experienced a decrease compared to their ranges at the end of therapy but their mobility was still better than their measurements before therapy. Five patients experienced no long-term improvement.

In head-and-neck cancer patients, radiation-induced fibrosis can lead to many different complaints, depending on the size and placement of treatment fields, the total dose, and whether the patient also had surgery. Limitations in neck range-of-motion are common and are quantifiable. Because this study was looking for objectively measured changes associated with microcurrent therapy, the protocol was designed to achieve improvement in range of motion. Measurements were made on all patients in the study regardless of whether the patient considered range-of-motion limitations to be a problem. In fact, most of the patients in the mildly and moderately limited groups had learned to compensate for the limitations and were surprised when measurements showed how much capability they had lost. As could be expected, the patients who were most severely limited received the greatest degree of benefit.

Patients also received relief from a number of complaints that were not directly targeted in the treatment protocol, the most significant of which were trismus and xerostomia. When the study was completed some case studies were done using a different microcurrent protocol along with physical therapy for the relief of trismus. The results were encouraging, and suggest that further studies on the role of microcurrent therapy in treating trismus are warranted.

Perhaps the most encouraging outcome of this study is the fact that many of the benefits observed at the end of the treatment week were sustained. In some cases there was continued improvement during the three-month follow-up period suggesting that the treatment had initiated tissue repair. Exact mechanisms for tissue repair are not completely understood, but one theory indicates that microcurrent stimulation influences the migration of extracellular calcium ions to penetrate the cell membrane. The higher level of intracellular calcium encourages increased synthesis of adenosine triphosphate (ATP). Protein synthesis is encouraged by affecting mechanisms that control DNA, thus encouraging cellular repair and replication. It is also believed that microvoltage may affect the cascade of reactions involved in a variety of inflammatory responses. Our data support the view that microcurrent therapy can initiate long-term benefit for patients suffering from fibrosis.

At the onset of the study it was expected that any improvement in symptoms would have been transient because no follow-up treatment was offered. The data indicate that this assumption was incorrect. Though the group size is small, data shown in FIGS. 5-7 suggest that improvement continued during the first and second month after microcurrent therapy. The treatment schedule needs to be optimized, perhaps delivering fewer treatments the first week followed by weekly and then monthly treatments to determine the maximum achievable benefit. For patients who are just beginning radiation therapy, it is possible that an optimum treatment schedule would include administering impedance-controlled microcurrent treatment concurrent with radiation therapy.

In designing the study the inventor deliberately excluded the use of any agent or activity that could contribute to relief of symptoms associated with fibrosis. Since this study has shown benefits attributable to microcurrent therapy alone, it is appropriate to consider combining this therapy with other physical therapy techniques or medications such as pentoxifylline/Vitamin E. Seven of the patients who benefited from microcurrent therapy indicated that they had received no benefit from previous physical therapy, but it is possible that the combination might be more effective than either single modality.

1. A system for monitoring range of motion obtainable by a patient, comprising:
two large protractors including circular scales and suspended in perpendicular planes above and in front of a patient measurement location, wherein said two large protractors are adapted for equidistantly positioning a patient's head with respect to said protractors in a manner that the patient is generally centered and positioned in front and beneath each protractor;
a head mountable harness including securing means adapted for temporary attachment of a laser about said head mounted harness; and
at least one laser adapted for mounting about said head mountable harness and capable of illuminating a laser beam onto said circular scales;
wherein said laser beam is used to indicate the range of motion of the patient's head based on said laser beam's location along the circular scale of at least one of said two large protractors.

2. The system of claim 1, wherein said head mountable harness further comprises at least one elastic band.

3. The system of claim 2, wherein said securing means comprises Velcro™.

4. The system of claim 2,-wherein said securing means comprises Velcro™.

5. The system of claim 1, wherein said circular scales include unit markings.

6. The system of claim 5, wherein said unit markings are in the format of degrees.

7. The system of claim 6 wherein movement of a patient's head causes movement of said laser beam along at least one of said two large protractors, said movement enabling measurement of a patient's ROM in degrees.

8. The invention of claim 1, wherein at least one of cervical rotation, extension-flexion and lateral-flexion can be measured for mobility of a patient's head and neck area.

9. A range-of-motion (ROM) measuring system, comprising:
at least one large protractor having an outer and an inner surface and including a circular scale including a vertex located along said at least one large protractor's inner surface, said at least one large protractor selectively suspendable above and in front of a patient measurement location, wherein said at least one large protractor adapted for equidistantly positioning a patient's head away with respect to said circular scale of said at least one large protractor in a manner that the patient is generally centered and positioned comfortably in front or beneath the vertex of said at least one large protractor;
a head mountable harness including securing means adapted for temporary attachment of a laser about said head mounted harness; and
at least one laser adapted for mounting about said head mountable harness and capable of illuminating a laser beam onto said circular scale;
wherein said laser beam is used to indicate the range of motion of the patient's head based on said laser beam's location indicated along said circular scale.

10. The invention of claim 1, wherein at least one of cervical rotation, extension-flexion and lateral-flexion can be measured for mobility of a patient's head and neck area.

11. The system of claim 9, wherein said head mountable harness further comprises at least one elastic band.

12. The system of claim 11, wherein said securing means comprises Velcro™.

13. The system of claim 9, wherein said securing means comprises Velcro™.

14. The system of claim 9, wherein said circular scale include unit markings in the format of degrees.

15. The system of claim 14 wherein movement of a patient's head causes movement of said laser beam along at least one of said two large protractors, said movement enabling measurement of a patient's ROM in degrees.

16. Methods of monitoring range-of-motion (ROM) by a patient, comprising the steps of:
positioning a patient's head equidistantly away from and at the center of a circular scale associated with at least one large protractor having an outer and an inner surface and including the circular scale on its inner surface, said circular scales further including a vertex, wherein said at least one large protractor is suspendable above or in front of said patient and wherein said at least one large protractor is adapted for location of the patient in a manner that the patient is generally centered and positioned at null comfortably in front or beneath the vertex of said at least one large protractor;
mounting a head mountable harness including securing means adapted for temporary attachment of at least one laser;
mounting at least one laser capable of illuminating a laser beam onto said circular scale onto said head mountable harness, wherein said laser beam can indicate the range of motion of the patient's head based on said laser beam's location indicated along said circular scale;
adjust said at least one laser whereby it is positioned relative to the point(s) about which the patient's head should pivot during at least one of: cervical rotation, extension-flexion and lateral-flexion, a further positioning said at least one laser such that the beam is intersects the vertex of the large protractors while said patient is positioned at null;
directing the patient to rotate, extend or laterally move the head;
recording positions along said circular scale that represent the patient's maximum ability to move the head.

17. The method of claim 17, wherein ROM measurements can be obtained before, during and after therapy.

18. The method of claim 18, wherein periodic patient positioning accuracy of about ±0.25 cm can be achieved when the distance of said circular scale from the patient's head is set at about 112 cm.

19. The methods of claim 16, including at least one of: monitoring the range of movement of an area of a cancer patient's body associated with microcurrent therapy.
